(19) United States
(12) Patent Application Publication
(10) Pub. No.: US 2019/0282158 A1
(43) Pub. Date: Sep. 19, 2019

(54) FALL MANAGEMENT USING A BLADDER SENSOR

(71) Applicant: Curbell Medical Products, Inc., Orchard Park, NY (US)

(72) Inventors: Donald J. Enzinna, Lockport, NY (US); Karen A. Clemens, East Aurora, NY (US)

(21) Appl. No.: 15/922,647

(22) Filed: Mar. 15, 2018

(51) Int. Cl. A61B 5/20; A61B 5/00

(52) U.S. Cl. CPC A61B 5/204; A61B 5/03; A61B 5/205; A61B 5/746

(57) ABSTRACT

A system for fall management is provided, including a bladder sensor configured to measure an amount of urine in a bladder of an individual; a controller configured to retrieve the amount of urine from the bladder sensor, determine if the amount of urine in the bladder has reached a threshold, and transmit a warning signal when the amount of urine reaches a threshold; a monitor, having a processor and a memory, configured to receive the warning signal transmitted by the controller; and alert a caregiver when the warning signal is received.

### FIELD OF THE DISCLOSURE

The present disclosure relates to patient monitoring, and more particularly to monitoring patients who may be at risk for falls.

### BACKGROUND OF THE DISCLOSURE

A great deal of effort and resources have been applied to the development and production of systems that help prevent falls in individuals having, for example, limited mobility. One strategy is to detect when an individual leaves a chair or bed to automatically summon a caregiver to assist the individual. A common system of this type employs a pressure pad placed under the individual. As long as the individual applies weight to the pad, the alarm is silent. When the individual attempts to stand and the pad is no longer under the weight of the individual, an alarm sounds. This type of system often falls short of the goal of fall prevention because the individual is already standing and at risk for a fall by the time the alarm has sounded.

There has been progress in building systems that detect movement earlier to allow a caregiver to attend to the individual earlier. These systems tend to detect motion, typically with video analytics. Systems based on video have a direct tradeoff with higher sensitivity and earlier warnings leading to a greater incident of false alarms. A second objection often arises due to the presence of a video camera continually watching the individual, regardless of whether the video is available for remote viewing or used only to generate a simple on or off alarm signal.

Accordingly, there is a critical, long-felt need for a predictive fall management system which anticipates that an individual is about to attempt to stand, rather than a reactive system which only alerts the caregiver when the individual has already begun standing.

### BRIEF SUMMARY OF THE DISCLOSURE

The present disclosure is based on the observation that, in many cases, when an individual exits a bed or chair, they do so because of the urge to use to the bathroom—i.e., to void their bladder. Accordingly, if it can be determined when the individual has the urge to void, it can also be deduced that the individual has a high probability of attempting to exit the bed or chair.

In an aspect of the present disclosure, a system for fall management is provided, including a bladder sensor configured to measure an amount of urine in a bladder of an individual. The system further includes a controller configured to retrieve the amount of urine from the bladder sensor, determine if the amount of urine in the bladder has reached a threshold, and transmit a warning signal when the amount of urine reaches a threshold. The system further includes a monitor, having a processor and a memory, configured to receive the warning signal transmitted by the controller, and alert a caregiver when the warning signal is received.

In some embodiments, the bladder sensor is wearable by the individual. In some embodiments, the bladder sensor is an infrared heart rate variability (HRV) sensor. In some embodiments, the bladder sensor is a distension sensor configured to measure curvature of a portion of skin adjacent to the bladder. In some embodiments, the bladder sensor is a bioelectrical impedance sensor configured to measure the conductivity of the urine in the bladder.

In some embodiments, the bladder sensor is powered by one or more batteries. In some embodiments, the warning signal is transmitted via one or more of a Bluetooth connection, a Zigbee connection, or a Wi-Fi connection. In some embodiments, the monitor is a mobile computing platform, such as a smartphone or tablet. In some embodiments, the monitor is a remote patient monitoring station.

In an aspect of the present disclosure a method for fall management is provided, including measuring, by a bladder sensor, an amount of urine in a bladder of an individual. The method further includes retrieving from the bladder sensor, by a controller, the amount of urine in the bladder. The method further includes determining, by a controller, when the amount of urine in the bladder has reached a threshold. The method further includes transmitting, by a controller, a warning signal when the amount of urine reaches a threshold. The method further includes receiving, by a monitor, the warning signal transmitted by the controller. The method further includes alerting, by the monitor, a caregiver when the monitor receives the warning signal.

### DESCRIPTION OF THE DRAWINGS

For a fuller understanding of the nature and objects of the disclosure, reference should be made to the following detailed description taken in conjunction with the accompanying drawings, in which:

FIG. 1 is a block diagram showing an embodiment of the present disclosure; and

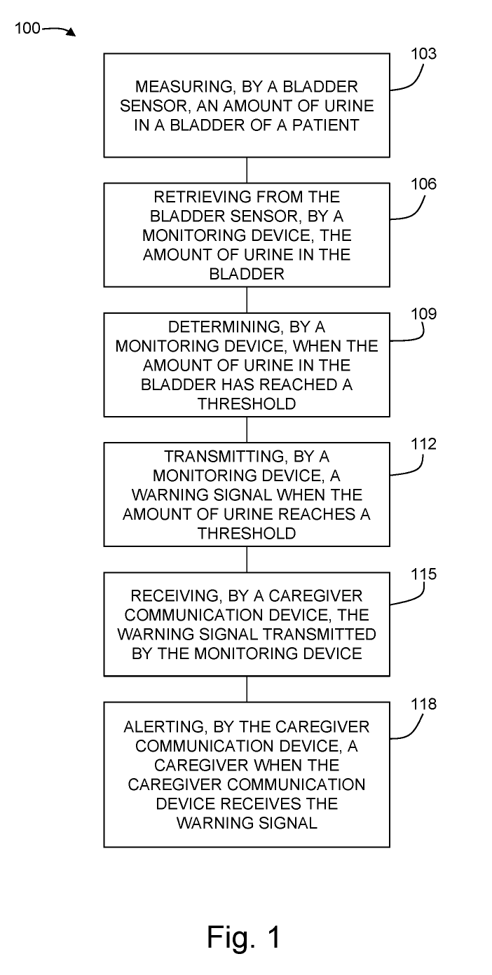

FIG. 2 is a diagram showing an embodiment of the present disclosure.

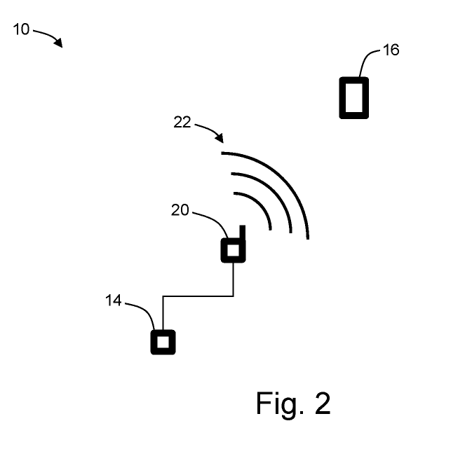

### DETAILED DESCRIPTION OF THE DISCLOSURE

In an aspect of the present disclosure, a system 10 for fall management is provided, including a bladder sensor 14 configured to detect a bladder status of an individual. For example, the bladder sensor 14 may be configured to detect an amount of urine in the bladder. The system 10 further includes a controller 20 configured to retrieve the bladder status from the bladder sensor 14, determine if the bladder status indicates a fall potential, and transmit a warning signal 22 if the bladder status is determined to indicate a fall potential. For example, a fall potential may be indicated if the bladder status indicates a full bladder of the individual (e.g., an amount of urine that exceeds a predetermined threshold). The system 10 further includes a monitor 16 configured to receive the warning signal 22 transmitted by the controller 20, and alert a caregiver if the warning signal 22 is received.

In some embodiments, the bladder sensor 14 is wearable by the individual 90.

In some embodiments, the bladder sensor 14 is an infrared heart rate variability (HRV) sensor. The HRV sensor measures the heart rate of an individual over a period of time to calculate the individual's HRV. The individual's HRV is then used to determine the fullness of the individual's bladder.

In some embodiments, the bladder sensor 14 is a distension sensor. The distension sensor may be configured to measure curvature of a portion of skin adjacent to the bladder. The curvature of the portion of the skin adjacent to the bladder is directly varied with the fullness of the bladder. The distension sensor may be located on a waistband of a pant or other garment worn by the individual.

In some embodiments, the bladder sensor 14 is a bioelectrical impedance sensor configured to measure the conductivity of the urine in the bladder.

Other embodiments of the present disclosure may include other types of bladder sensors using a wide variety of sensing means other than the sensors disclosed above. These bladder sensors are traditionally marketed either to individuals with nerve damage where the natural urge to urinate may be impaired and result in overfilling of the bladder, or to parents of children being potty-trained.

In some embodiments, the bladder sensor 14 is powered by one or more batteries.

In some embodiments, the warning signal 22 is a wireless signal. For example, the wireless signal may be transmitted via a Bluetooth® connection. In other examples, the wireless may be transmitted via Zigbee® or Wi-Fi connection. The warning signal 22 may be transmitted by more than one mode of wired or wireless signals.

In some embodiments, the monitor 16 is a mobile computing platform, such as a smartphone, tablet, or any suitable portable device. In a further embodiment, the mobile computing platform may connect to a battery-powered bladder sensor 14 via Bluetooth connection. An app running on the mobile computing platform may remind the individual to void before overfilling of the bladder can occur.

In some embodiments, the monitor 16 is a remote patient monitoring station.

In an aspect of the present disclosure a method 100 for fall management is provided. The method includes detecting 103 a bladder status of an individual. The method 100 further includes determining 106 a fall potential based on the detected bladder status. The method 100 further includes alerting 109 a caregiver of a determined 106 fall potential. For example, a caregiver may be alerted by transmitting 112 a warning signal to a monitor, for example, a remote patient monitoring station. In this way, a caregiver may be alerted by, for example, an audible alarm.

In an exemplary embodiment of the present disclosure, an individual 90 wears a bladder sensor 14 which reports to a controller 20. The controller 20 continuously polls the bladder sensor 14 and measures the amount of urine in the individual's bladder. When a threshold is reached, an alert is issued to a caregiver that the individual has a full bladder and will need to use the bathroom soon. The caregiver can then preemptively approach the individual and ask if they want to go to the bathroom well before the individual has the urge to get up and go by themselves. In the case of the battery operated, Bluetooth-ready bladder sensor 14, this can be incorporated into the hospital infrastructure, for example, a remote patient monitoring station of a nurse station, using an in-room communication device, such as, for example, a Rego communication device.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof

We claim:

1. A system for fall management, comprising:
a bladder sensor configured to detect a bladder status of an individual;
a controller in communication with the bladder sensor, the controller configured to:
retrieve the bladder status from the bladder sensor;
determine if the bladder status indicates a fall potential; and
transmit a warning signal if the bladder status indicates a fall potential; and
a monitor, configured to:
receive the warning signal transmitted by the controller; and
alert a caregiver.

2. The system of claim 1, wherein the bladder sensor is wearable by the individual.

3. The system of claim 1, wherein the bladder sensor is an infrared heart rate variability sensor.

4. The system of claim 1, wherein the bladder sensor is a distension sensor.

5. The system of claim 4, wherein the distension sensor is configured to measure a curvature of a portion of skin adjacent to the bladder.

6. The system of claim 1, wherein the bladder sensor is a bioelectrical impedance sensor configured to measure a conductivity of the urine in the bladder.

7. The system of claim 1, wherein the bladder sensor is powered by one or more batteries.

8. The system of claim 1, wherein the warning signal is a wireless signal.

9. The system of claim 8, wherein the warning signal is transmitted via one or more of a Bluetooth connection, a Zigbee connection, and a Wi-Fi connection.

10. The system of claim 1, wherein the monitor is a mobile computing platform.

11. The system of claim 1, wherein the monitor is a remote patient monitoring station.

12. The system of claim 1, wherein the bladder detects an amount of urine in a bladder of the individual.

13. The system of claim 12, wherein the controller determines a likelihood of an urge to void by determining if the amount of urine is greater than a threshold.

14. A method for fall management, comprising:
detecting a bladder status of an individual;
determining a fall potential based on the detected bladder status;
alerting a caregiver if a fall potential is determined.

15. The method of claim 14, further comprising transmitting a warning signal to a monitor.
